(19) United States
(12) Patent Application Publication
(10) Pub. No.: US 2025/0313898 A1
(43) Pub. Date: Oct. 9, 2025

(54) SINGLE MOLECULE SEQUENCING AND METHYLATION PROFILING OF CELL-FREE DNA

(71) Applicant: The Board of Trustees of the Leland Stanford Junior University, Stanford, CA (US)

(72) Inventors: Billy Tsz Cheong LAU, San Mateo, CA (US); Hanlee P. JI, Stanford, CA (US)

(21) Appl. No.: 18/867,415

(22) PCT Filed: May 31, 2023
(86) PCT No.: PCT/US2023/023970

§ 371 Date: Nov. 19, 2024

Related U.S. Application Data
(60) Provisional application No. 63/348,425, filed on Jun. 2, 2022.

(51) Int. Cl. C12Q 1/6886; C12N 15/10

(52) U.S. Cl. CPC C12Q 1/6886; C12N 15/1065; C12Q 2600/154

(57) ABSTRACT

The disclosure provides methods for detecting a molecule of tumor DNA (tDNA) in a sample of cell-free DNA (cfDNA). In certain embodiments, cfDNA is sequenced using a single molecule sequencing to obtain a methylation profile of a sequence read. Such methylation profile is compared to a reference methylation profile from a cancer cell and/or a non-cancer cell to identify the sequence read as being from a molecule of tDNA. Further embodiments provide estimating the number of molecules of tDNA in the sample of cfDNA and, to determine as a tumor load of the cfDNA, the proportion of the number of molecules of tDNA to the total number of molecules of cfDNA in the sample. Such tumor load can be used to monitor cancer progression in a subject or efficacy of a cancer therapy administered to a subject.

### CROSS-REFERENCING

This application claims the benefit of U.S. provisional application Ser. No. 63/348,425, filed on Jun. 2, 2022, which application is incorporated by reference herein for all purposes.

### INTRODUCTION

Malignant tumor cells shed their DNA into the bloodstream of cancer patients. Sequencing the cell-free DNA (cfDNA) identifies somatic mutations and copy number changes; this approach is referred to as a liquid biopsy. Epigenetic modifications of tumor DNA are of particular interest for their role in tumorigenesis and progression. Characterizing these cancer-specific methylation changes from circulating tumor DNA (ctDNA) has proven to be a highly sensitive and specific modality for liquid biopsies. DNA is typically processed with bisulfite or enzymatic conversion of unmodified cytosines into uracil bases for Illumina-based methylation detection, followed by sequencing with an Illumina system. However, this approach introduces biases such as significant GC skews and oxidative DNA damage, with substantial impacts on PCR amplification biases and alignment artifacts. Overall, characterizing methylated cfDNA from cancer patients with conventional approaches remains a challenge.

### SUMMARY

Epigenetic characterization of cfDNA is a rapidly emerging field for liquid biopsy characterization. This disclosure provides a process for high-throughput sequencing of cfDNA on single molecule sequencers, (e.g., Oxford Nanopore, Pacific Biosciences), which enables yields from millions to hundreds of millions of reads per sample. The genome-wide methylation profiles of cancer patient-derived cfDNA was identified. By using matched tumors and other sample types, such as blood, as a methylation reference, the methods disclosed in this disclosure enable detecting ctDNA and/or to determine the load of ctDNA in cfDNA of a subject. The load of ctDNA in a cfDNA sample from a subject can be used for detecting cancer, monitoring of tumor burden, for example, to monitor disease progression or efficacy of a cancer therapy.

The present method allows on to characterize methylation patterns from cell-free DNA isolated from body fluids, particularly from cancer patients, without PCR (FIG. 1A). This approach is believed to overcome some of the potential problems with conventional methylation sequencing of cfDNA. The methods disclosed herein comprise characterizing methylated DNA without any chemical or enzymatic conversion, as required with short-read approaches. Moreover, the present methods do not utilize PCR amplification, thus enabling single-molecule counting of cfDNA molecules without UMI (unique molecular index) barcodes. Methylated DNA generates a unique single molecule sequencing signal compared to unmodified DNA, and is readily detected with various machine learning algorithms. Therefore, single molecule sequencing methylation profiles directly reflect the native state of the cfDNA without the typical skews and biases introduced through conventional methods of DNA sequencing preparation.

While single molecule sequencing often requires hundreds of nanograms of genomic DNA, single molecule sequencing of cfDNA is herein demonstrated with one to five nanograms or less per sample. To that end, experimental parameters were optimized to maximize the yield of ligation reactions of the sample barcode and single molecule sequencing adaptors to cfDNA (FIG. 3). Sequencing libraries derived from nucleosomal DNA were created for initial tests, modeling the pattern of DNA fragmentation occurring in blood. Using open source analysis packages (FIG. 4), single molecule sequencing identified tens of millions of methylated sites, with values corresponding to observed methylation percentage. Sequencing libraries were also generated from the same DNA mixtures using conventional protocols for library preparation. Here, a median improvement of about an order of magnitude was observed in aligned reads utilizing input amounts greater than 100 pg, enabling high-throughput sequencing of cfDNA (FIG. 1B).

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1G. Single molecule sequencing of cfDNA. (A) An optimized protocol for generating sequencing libraries cfDNA libraries enables high-throughput methylation characterization. (B) Cell free DNA library comparison. An optimized workflow enables about an order of magnitude increase in sequencing yield versus a conventional protocol. (C) Sequencing yield correlation with input cfDNA. Fluorometric quantification was performed on cancer patient-derived cfDNA samples, and compared to the aligned sequencing yield. (D) Nucleosome profiles of healthy and patient-derived cfDNA. Fragment sizes of cfDNA were estimated by using the aligned sequence length, and plotted for cfDNA from four healthy donors and 20 colorectal cancer patients. (E) Genome-wide methylation quantification. Methylation across the genome was computed for healthy and patient-derived cfDNA. (F) Nucleosome enrichment analysis. The ratio of mono-nucleosomes to di-nucleosomes was quantified for each cell type. (G) Methylation profiles of healthy- and patient-derived cfDNA. Gene-level methylation values for each sample were determined, and statistically significant ones are plotted and clustered as a heatmap.

FIGS. 2A-2D. Single-molecule methylated sequence classification. (A) Overview of method. Reads are classified alongside a set of candidate sample reference methylomes to determine a potential matching sample type. Sites are merged between the aligned read and candidate methylome, after which methylation states are compared. (B) Classification accuracy. GP2D and healthy donor-derived nucleosome mixtures were used to validate the classification procedure. ROC curves are plotted, where each curve represents a distinct immune threshold score. The curve is plotted by varying the cancer threshold score. (C) Admixture validation. The proportion of reads classified as belonging to cell line reference is plotted as a function of the actual admixture ratio and sequencing depth. (D) Longitudinal methylation profiles of patient-derived cfDNA. The overall cfDNA sequencing yield (top) is plotted against the number of reads with methylation profiles matching the primary tumor with a tumor score of >0.9 (bottom). Clinically relevant events were annotated.

FIG. 3. Schematic representation of optimized cfDNA library preparation protocol.

FIG. 4. Schematic representation of sequencing data analysis.

FIG. 5. Gene list enrichment analysis showing significant hits in the Myc pathway.

FIG. 6. A dual-threshold score to stringently classify individual cfDNA reads as immune- or cancer-derived.

FIG. 7. Variation in accuracy based on different stringency thresholds for classification of cfDNA as immune-derived or cancer-derived.

FIG. 8. The correlation between the stringency cutoff criteria and the proportion of reads that can be confidently classified as immune-derived or cancer-derived.

FIG. 9. Experimental and bioinformatics steps of certain exemplary methods disclosed herein.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

A “subject” or “patient” as used herein can be a human or a non-human animal. A non-human animal can be a primate, a canine, a feline, a bovine, or an equine animal.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112. In describing and claiming the present invention, certain terminology will be used in accordance with the definitions set out below. It will be appreciated that the definitions provided herein are not intended to be mutually exclusive.

As used herein, the phrases “for example,” “for instance,” “such as,” or “including” are meant to introduce examples that further clarify more general subject matter. These examples are provided only as an aid for understanding the disclosure and are not meant to be limiting in any fashion.

As used herein, the terms “may,” “optional,” “optionally,” or “may optionally” mean that the subsequently described circumstance may or may not occur, so that the description includes instances where the circumstance occurs and instances where it does not.

Definitions of other terms and concepts appear throughout the detailed description.

### DETAILED DESCRIPTION

Single molecule sequencing, such as what may be conducted with instruments such as Oxford Nanopore or Pacific Biosciences sequencing of cfDNA for measuring tumor burden is demonstrated. Despite the overall sequencing yield being orders of magnitude below what is achievable with Illumina sequencing, single molecule sequencing offers significant advantages compared to short-read approaches. Measuring DNA methylation with short read sequencing such as an Illumina sequencer requires extensive sample manipulation, amplification, and bioinformatic processing. This disclosure demonstrates that streamlined methylation analysis of cfDNA is feasible with significantly fewer experimental procedures and bottlenecks. As single molecule-based cfDNA methylation analysis is only dependent on machine learning models rather than on experimental manipulation of unmethylated residues, newer models can be applied to archived raw data to incorporate the detection of other modified bases. Methylation profiling of cfDNA has previously been shown to identify correlative features such as tissue-of-origin, gene expression, and tumor subtyping-single molecule sequencing, by the virtue of native DNA processing, will help accelerate this process. In summary, the methods disclosed herein can significantly expand on epigenomic analysis of cell-free DNA, which can significantly impact liquid biopsy-based diagnosis for cancer as well as monitoring of disease progression or efficacy of a cancer therapy administered to a subject.

Certain embodiments of the disclosure provide a method for detecting a molecule of circulating tumor DNA (ctDNA) in a sample of cell-free DNA (cfDNA). The method comprises sequencing the sample of cfDNA using a single molecule sequencing to obtain sequence reads.

A sequencing read so obtained is analyzed by:
(a) identifying in the sequence read differentially methylated CpG sites, the differentially methylated CpG sites having different methylation status in a cancer cell versus a non-cancer cell,
(b) determining which differentially methylated CpG sites in the sequence read are methylated and which differentially methylated CpG sites are unmethylated to obtain a methylation profile for the sequence read;
(c) calculating a first methylation score based on: i) the number of differentially methylated CpG sites in the sequence read that matches the methylation status of the differentially methylated CpG sites in the cancer cell and ii) the total number of differentially methylated CpG sites in the sequence read,
(d) calculating a second methylation score based on: i) the number of differentially methylated CpG sites in the sequence read that matches the methylation status of the differentially methylated CpG sites in a non-cancer cell and ii) the total number of differentially methylated CpG sites in the sequence read,
(e) identifying the sequence read as being derived from a cancer cell based the scores calculated in steps (c) and (d).

A “differentially methylated CpG site” as used herein refers to a CpG site that differs in its methylation status between a cancer cell versus a non-cancer cell. A differentially methylated CpG site can be identified based on the genomic co-ordinates of the CpG site. For example, a differentially methylated CpG site in a human can be identified based on its co-ordinates in the human genome, for example, in the GRCh38 reference human genome.

A differentially methylated CpG site is methylated in a cancer cell and non-methylated in a non-cancer cell. Alternatively, a differentially methylated CpG site is non-methylated in a cancer cell and methylated in a non-cancer cell. A CpG site that is methylated in both a cancer cell and a non-cancer cell is not a differentially methylated CpG site. Similarly, a CpG site that is unmethylated in both a cancer cell and a non-cancer cell is not a differentially methylated CpG site.

A CpG site can also be partially methylated, with methylation values in between 100% (methylated) and 0% (non-methylated) methylation. Thus, a differentially methylated site is also identified as a partially methylated site where the methylation value differs between a cancer cell versus a non-cancer cell.

Owing to the difference in the methylation status of a differentially methylated CpG site in a cancer cell versus a non-cancer cell, the differentially methylated CpG site can be used to identify a sequence read from a cfDNA as being from a molecule of tumor DNA (DNA) based on the methylation status of the differentially methylated CpG site. For example, if the methylation status of a differentially methylated CpG site in a sequence read matches with the methylation status of that CpG site in a cancer cell, then the sequence read can be identified as being from a molecule of tDNA. Alternatively, if the methylation status of a differentially methylated CpG site in a sequence read matches with the methylation status of that CpG site in a non-cancer cell, then the cfDNA can be identified as not being from a molecule of tDNA.

A methylation profile of differentially methylated CpG sites in a cancer cell and a non-cancer cell can be determined based on the comparison of the methylation status of the differentially methylated CpG sites in the cancer cell and the non-cancer cells. The CpG sites that differ in their methylation status between the cancer and non-cancer cells can then be identified as differentially methylated CpG sites.

The methods disclosed herein comprise determining which differentially methylated CpG sites in the sequence read are methylated and which differentially methylated CpG sites are unmethylated to obtain a methylation profile for the sequence read. To determine in a sequence read which CpG sites are differentially methylated CpG sites, the sequence read can be aligned to a genomic region. Then, the differentially methylated CpG sites in that genomic region can be identified based on the methylation profiles of differentially methylated CpG sites in a cancer cell and a non-cancer cells.

In some cases, the differentially methylated CpG sites are specific to a tissue, for example, brain, breast, pineal gland, pituitary gland, thyroid gland, parathyroid glands, thorax, heart, lung, esophagus, thymus gland, adrenal glands, appendix, gall bladder, urinary bladder, large intestine, small intestine, kidneys, liver, pancreas, spleen, stoma, ovaries, uterus, testis, skin, or blood.

In some cases, the differentially methylated CpG sites are specific to a cancer type. A cancer can be a cancer of hematological origin, brain cancer, breast cancer, lung cancer, gastrointestinal cancer, head and neck cancer, cervical cancer, liver cancer, skin cancer, uterine cancer, etc. Additional cancer types are known in the art and use of the methods disclosed herein for analyzing such cancers is within the purview of the disclosure.

In certain single molecule sequencing methods, as each molecule is being sequenced, methylated DNA generates a unique signal (either optical imaging or electrical detection) compared to unmodified DNA. Thus, such single molecule sequencing methods not only determine the DNA sequence but also determine the methylation status of nucleotides within the sequence. The single molecule sequencing methods that can be used in the methods disclosed herein include nanopore sequencing or single molecule real-time (SMRT) sequencing.

The methylation status of the differentially methylated CpG sites in a sequence read is used to determine a methylation profile for the sequence read. Thus, a methylation profile of a sequence read provides methylation status of differentially methylated CpG sites in the sequence read.

The methylation profile of a sequence read can be used to calculate a first methylation score based on: i) the number of differentially methylated CpG sites in the sequence read that matches the methylation status of the differentially methylated CpG sites in the cancer cell and ii) the total number of differentially methylated CpG sites in the sequence read.

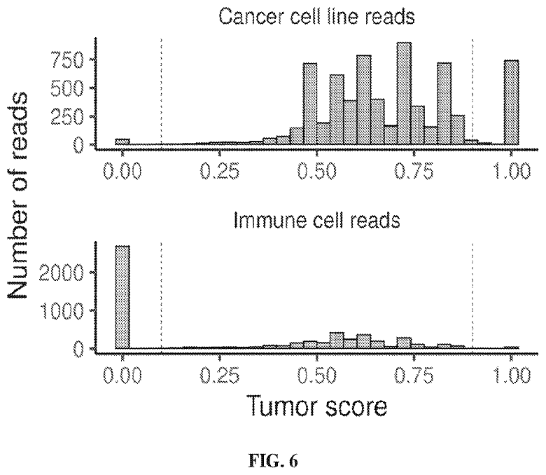

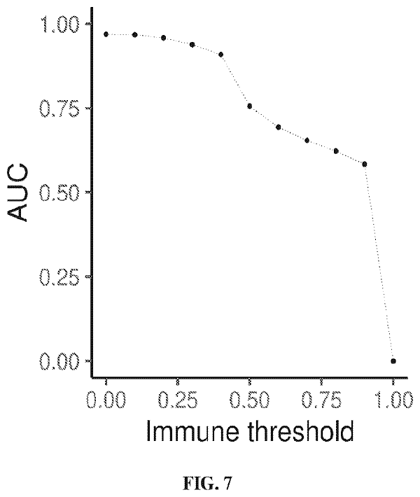

Thus, the first methylation score indicates the extent of similarity between methylation status of differentially methylated CpG sites in a sequence read with the methylation status of the differentially methylated CpG sites in a cancer cell. The first methylation score is also referenced in this disclosure as “tumor score.” An example of first methylation scores (tumor scores) for sequence reads from cancer cells is provided in FIG. 6.

For example, if a sequence read contains ten differentially methylated CpG sites and five of those CpG sites have the same methylation status as the differentially methylated CpG sites in a cancer cell, then a first methylation score can be 0.5 or 50%, i.e., the ratio or percentage of differentially methylated CpG sites in the sequence read that matches the methylation status of differentially methylated CpG sites in a cancer cell.

Similarly, if a sequence read contains ten differentially methylated CpG sites and nine of those CpG sites have the same methylation status as the differentially methylated CpG sites in a cancer cell, then a first methylation score can be 0.9 or 90%, i.e., the ratio or percentage of differentially methylated CpG sites in the sequence read that matches the methylation status of differentially methylated CpG sites in a cancer cell.

The methylation profile of a sequence read can also be used to calculate a second methylation score based on: i) the number of differentially methylated CpG sites in the sequence read that matches the methylation status of the differentially methylated CpG sites in the non-cancer cell and ii) the total number of differentially methylated CpG sites in the sequence read.

Thus, the second methylation score indicates the extent of similarity between methylation status of differentially methylated CpG sites in a sequence read with the methylation status of the differentially methylated CpG sites in a non-cancer cell. An example of first methylation scores (tumor scores) for sequence reads from non-cancer cells (normal immune cells) is provided in FIG. 6.

For example, if a sequence read contains ten differentially methylated CpG sites and five of those CpG sites have the same methylation status as the differentially methylated CpG sites in a non-cancer cell, then a second methylation score can be 0.5 or 50%, i.e., the ratio or percentage of differentially methylated CpG sites in the sequence read that matches the methylation status of differentially methylated CpG sites in a non-cancer cell.

Similarly, if a sequence read contains ten differentially methylated CpG sites and nine of those CpG sites have the same methylation status as the differentially methylated CpG sites in a non-cancer cell, then a second methylation score can be 0.9 or 90%, i.e., the ratio or percentage of differentially methylated CpG sites in the sequence read that matches the methylation status of differentially methylated CpG sites in a non-cancer cell.

The first and the second methylation scores can be used to identify a sequence read as being from a molecule of tDNA. Various calculations and/or comparisons can be used to identify a sequence read as being or not being from a molecule of tDNA based on the first and the second methylation scores.

For example, a sequence read can be identified as being from a molecule of tDNA if the first methylation score is at or above a threshold. Such threshold can be from 0.5 to 1, such as 0.5, 0.6, 0.7, 0.8, 0.9, or 1.

For example, when the threshold is 0.5, a sequence read is identified as being from a molecule of tDNA if the first methylation score is 0.5 or above, i.e., at least half of the differentially methylated CpG sites in a sequence read have the same methylation status as that of a cancer cell. Alternatively, when the threshold is 0.8, a sequence read is identified as being from a molecule of tDNA if the first methylation score is 0.8 or above, i.e., at least 80% of the differentially methylated CpG sites in a sequence read have the same methylation status as that of a cancer cell.

Thus, higher first methylation score indicates higher likelihood that a sequence read is from a molecule of tDNA. Therefore, stringency of identifying a sequence read as being from a molecule of tDNA can be increased by setting a higher threshold of the first methylation score for identifying a sequence read as being from a molecule of tDNA.

A sequence read can be identified as not being from a molecule of tDNA if the second methylation score is at or above a threshold. Such threshold can be from 0.5 to 1, such as 0.5, 0.6, 0.7, 0.8, 0.9, or 1.

For example, when a threshold is 0.5, a sequence read is identified as not being from a molecule of tDNA if the second methylation score is 0.5 or above, i.e., at least half of the differentially methylated CpG sites in a sequence read have the same methylation status as that of a non-cancer cell. Alternatively, when a threshold is 0.8, a sequence read is identified as not being from a molecule of tDNA if the second methylation score is 0.8 or above, i.e., at least 80% of the differentially methylated CpG sites in a sequence read have the same methylation status as that of a non-cancer cell.

Thus, higher second methylation score indicates higher likelihood that a sequence read is not from a molecule of DNA. Therefore, stringency of identifying a sequence read as not being from a molecule of tDNA can be increased by setting a higher threshold of the second methylation score for identifying a sequence read as not being from a molecule of tDNA.

In some cases, the two thresholds are used to identify a sequence read as being or not being from a molecule of tDNA. For example, a sequence read is identified as being from a molecule of tDNA if the first methylation score is at or above a first threshold (e.g., 0.7 or above, 0.8 or above, or 0.9 or above) and the sequence read is identified as not being from a molecule of tDNA if the second methylation score is at or above a second threshold (e.g., 0.7 or above, 0.8 or above, or 0.9 or above).

In some cases, the two thresholds are numberically identical to each other, for example: the first threshold is 0.7 and the second threshold is also 0.7, the first threshold is 0.8 and the second threshold is also 0.8, or the first threshold is 0.9 and the second threshold is also 0.9.

In some cases, the two thresholds are numerically different from each other, for example: the first threshold is 0.7, 0.8, or 0.9 and the second threshold is 0.7, 0.8, or 0.9 but is different from the first threshold.

A sequence read is identified as being from tDNA only if the first methylation score is higher than a first threshold and a sequence read is identified as not being from tDNA only if second methylation score is higher than a second threshold. A sequence read which has the first methylation score below the first threshold (e.g., 0.7, 0.8, or 0.9) and the second methylation score below the second threshold (e.g., 0.7, 0.8, or 0.9) cannot be definitively identified as being or not being from a molecule of tDNA. In some cases, sequence reads that cannot be definitively identified as being or not being from a molecule of tDNA can be excluded in the analysis of the cfDNA sample, for example, in determining the tumor load of the cfDNA discussed below.

In some cases, the ratio of a first methylation score and the second methylation score can be used to identify a sequence read as being from a molecule of tDNA. For example, a sequence read is identified as being from a molecule of tDNA if the ratio of the first methylation score to the second methylation score is 1.25 or more, for example, 1.5, 2, 3, 4, 5, 6, 7, 8, 9, or 10 or more.

When the ratio of the first methylation score to the second methylation score is 2, it indicates that a sequence read has twice the number of differentially methylated CpG sites that matches their methylation status with a cancer cell as compared to the number of differentially methylated CpG sites that matches their methylation status with a non-cancer cell.

Similarly, when the ratio of the first methylation score to the second methylation score is 3, it indicates that a sequence read has thrice the number of differentially methylated CpG sites that matches their methylation status with a cancer cell as compared to the number of differentially methylated CpG sites that matches their methylation status with a non-cancer cell.

Thus, higher ratio of the first methylation score to the second methylation score indicates higher likelihood that a sequence read is from a molecule of tDNA. Therefore, stringency of identifying a sequence read as being from a molecule of tDNA can be increased by setting a higher threshold for the ratio of the first methylation score to the second methylation score for identifying a sequence read as being from a molecule of tDNA.

In some cases, the ratio of a second methylation score to the first methylation score can be used to identify a sequence read as not being from a molecule of tDNA. For example, a sequence read is identified as not being from a molecule of tDNA if the ratio of the second methylation score to the first methylation score is 1.25 or more, for example, 1.5, 2, 3, 4, 5, 6, 7, 8, 9 or 10 or more.

When the ratio of the second methylation score to the second methylation score is 2, it indicates that a sequence read has twice the number of differentially methylated CpG sites that matches their methylation status with a non-cancer cell as compared to the number of differentially methylated CpG sites that matches their methylation status with a cancer cell.

Similarly, when the ratio of the second methylation score to the first methylation score is 3, it indicates that a sequence read has about thrice the number of differentially methylated CpG sites that matches their methylation status with a non-cancer cell as compared to the number of differentially methylated CpG sites that matches their methylation status with a cancer cell.

Thus, higher ratio of the second methylation score to the first methylation score indicates higher likelihood that a sequence read is not from a molecule of tDNA. Therefore, stringency of identifying a sequence read as not being from a molecule of tDNA can be increased by setting a higher threshold for the ratio of the second methylation score to the first methylation score for identifying a sequence read as being from a molecule of tDNA.

In some cases, the fragmentation and size pattern of single molecule reads may be used to identify as being from a molecule of tDNA.

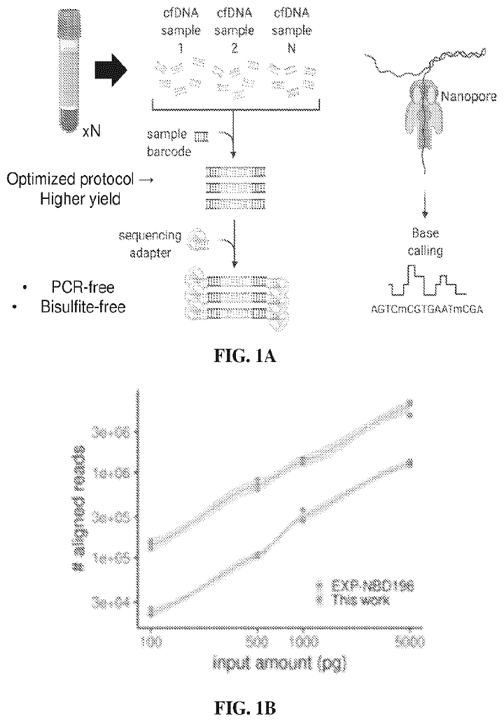

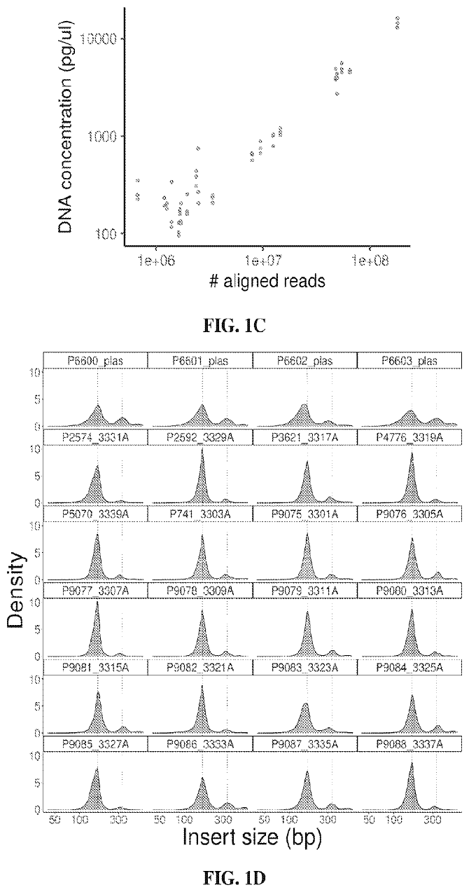

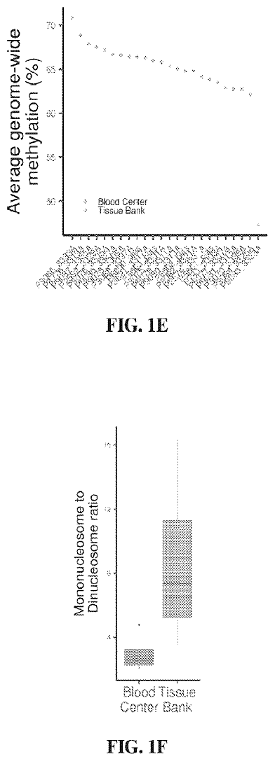

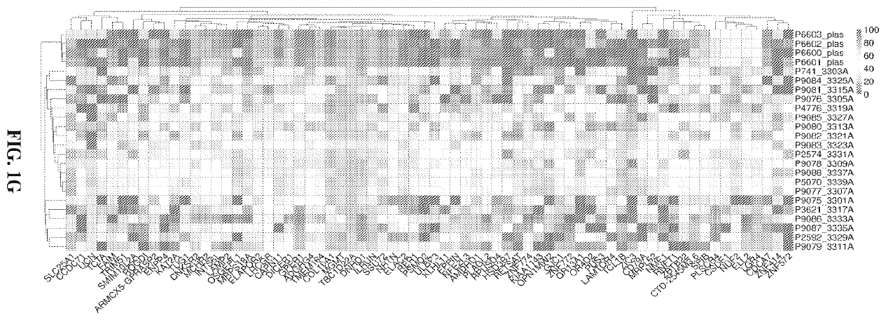

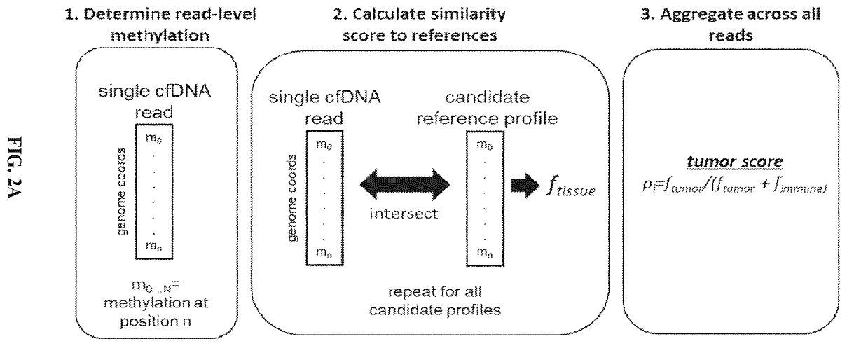

In certain single molecule sequencing methods, the actual size of a sequenced cfDNA molecule is identifiable from sequence alignment to the reference genome. The size and fragmentation pattern can be compared to patterns from cancer and non-cancer cells. An example of fragmentation and size patterns for cfDNA sequence reads from healthy donors and cancer patients is shown in FIG. 1F. In certain cases, cfDNA methylation can alter these fragmentation patterns and this joint information can provide additional characteristics to determine a disease state.

A single molecule read can be assigned to a mono- or di-nucleosome fragment size. Using a cutoff of 250 bp between mono- and di-nucleosome fragment sizes, the ratio between the number of mono-nucleosome and di-nucleosome cfDNA sequenced can be calculated.

In some cases, the fragment size of healthy and cancer cfDNA can be determined by the ratio between mono-nucleosome and di-nucleosome reads. Cancer patient-derived cfDNA may be enriched in certain nucleosome states. For example, cancer patient-derived cfDNA may be highly enriched in mono-nucleosomes versus di-nucleosomes. Other statistical properties such as the mean and variance in mono-nucleosome or di-nucleosome fragment size can be calculated.

By using a reference fragment distribution of cfDNA from healthy donors and cancer patients, fragmentation patterns from new samples can be matched and used to detect tDNA.

For example, a reference sequenced cohort of cfDNA from healthy and cancer patients may yield a mono-nucleosome to di-nucleosome ratio. This ratio can be from numbers from at least 1, such as 1, 2, 3, 4, 5, 6, 7, 8, 9, or at 10.

When a new cfDNA sample is sequenced, the mono-nucleosome to di-nucleosome ratio may be calculated. This ratio can also be from numbers of at least 1, such as 1, 2, 3, 4, 5, 6, 7, 8, 9, or at 10.

The mono-nucleosome to di-nucleosome ratio can be compared to the reference cohort using a statistical test or by a threshold to estimate tumor load. For example, the mono-nucleosome to di-nucleosome ratio of a new cfDNA sample can be classified as being similar to cancer cfDNA if it is above a certain threshold. Such threshold can be from at least 4, such as 4, 5, 6, 7, 8, 9, or 10. A higher ratio past the threshold can indicate a higher tumor load.

Any suitable sequencing technique can be used for single molecule sequencing used in the methods disclosed herein.

In some cases, the single molecule sequencing is nanopore-based sequencing. Alternatively, the single molecule sequencing is single molecule real time (SMRT) sequencing.

Certain details of SMRT (developed by Pacific Biosciences (PacBio)™) and single molecule sequencing (developed by Oxford Single molecule Technologies™) are described by the publication Logsdon et al. (2020), Long-read human genome sequencing and its applications, Nature Reviews Genetics, Vol. 21, pages 597-614, which is herein incorporated by reference in its entirety.

Briefly, in SMRT sequencing, an amplicon is ligated to hairpin adapters to form a circular molecule, called a SMRT bell. The SMRTbell is bound by a DNA polymerase and loaded onto a SMRT Cell for sequencing. A SMRT Cell can contain up to 8 million zero-mode waveguides (ZMWs). ZMWs are chambers of picolitre volumes. Light penetrates the lower 20-30 nm of SMRT Cells. The SMRTbell template and polymerase become immobilized on the bottom of the chamber. During the sequencing reaction, fluorescently labelled deoxynucleoside triphosphates (dNTPs) are incorporated into the newly synthesized strand, a fluorescent dNTP is held in the detection volume, and a light pulse from the well excites the fluorophore. A camera detects the light emitted from the excited fluorophore, which records the wavelength and the position of the incorporated base in the nascent strand. The DNA sequence is determined by the changing fluorescent emission that is recorded within each ZMW.

In nanopore sequencing, long DNA strand is tagged with sequencing adapters preloaded with a motor protein on one or both ends. The DNA is combined with tethering proteins and loaded onto the flow cell for sequencing. The flow cell contains protein nanopores embedded in a synthetic membrane. The tethering proteins bring the molecules to be sequenced towards the nanopore and as the motor protein unwinds the DNA, an electric current is applied, which drives the negatively charged DNA through the pore. The DNA is sequenced as it passes through the pore and causes characteristic changes in the current.

In certain embodiments, identifying a plurality of sequence reads from a cfDNA sample as being or not being from a molecule of tDNA can be used to estimate the number of molecules of tDNA in a sample of cfDNA. The proportion of tDNA molecules in a cfDNA sample can be used to estimate “tumor load” of the cfDNA sample.

In some cases, a tumor load is calculated as the percentage of sequence reads identified as being from a molecule of tDNA as compared to the total number of sequence reads in a cfDNA sample. For example, if one million sequence reads are produced from a cfDNA sample and 1,000 reads are identified as being from tDNA, then the tumor load of that cfDNA sample is 0.1%.

In some cases, a tumor load is calculated as the percentage of sequence reads identified as being from a molecule of tDNA as compared to the number of sequence reads in a cfDNA sample for which an identification is made. Thus, in this calculation, sequence reads that cannot be definitively identified as being or not being from a molecule of tDNA are ignored. For example, if a million sequence reads are produced from a cfDNA sample and 1,000 reads are identified as being from tDNA and 500,000 reads cannot be definitively identified as being or not being from a molecule of tDNA, then the tumor load of that cfDNA sample is 0.2%.

Higher percentage of tDNA molecules in a cfDNA sample indicates higher tumor load, which may indicate a more advanced disease or a higher number of cancer cells in a subject. Conversely, lower percentage of DNA molecules in a cfDNA sample indicates lower tumor load, which may indicate a more advanced disease or a lower number of cancer cells in a subject.

Thus, a tumor load of cfDNA sample from a subject can be used to estimate the disease status in a cancer patient. Such status can be used to diagnose cancer in a subject, monitor cancer progression in a subject, or monitor efficacy of a cancer therapy administered to a subject.

Accordingly, certain embodiments of the disclosure provide a method of diagnosing cancer in a subject by estimating a tumor load in the subject according to the methods disclosed herein and identifying the presence of cancer in the subject if the tumor load is at or above a threshold.

In some cases, the disclosure provides a method of monitoring cancer progression in a subject by estimating a tumor load in the subject according to the methods disclosed herein at a first time point and at a second time point, the first time point being earlier than the second time point. If the tumor load at the first time point is lower than the tumor load at the second time point, then the cancer is progressing in the subject. Also, the magnitude of increase from the first time point to the second time point would indicate the speed of cancer progression. A higher increase in the tumor load would indicate faster cancer progression, whereas a relatively lower increase in the tumor load would indicate a relatively slower cancer progression.

In some cases, the disclosure provides a method of monitoring cancer therapy in a subject by estimating a tumor load in the subject according to the methods disclosed herein at a first time point and at a second time point, the first time point being earlier than the second time point.

If the tumor load at the first time point is higher than the tumor load at the second time point, then the cancer therapy is effective in treating cancer in the subject. Also, the magnitude of decrease would indicate the efficacy of the cancer therapy. A bigger decrease in the tumor load would indicate more efficacious cancer therapy, whereas a relatively smaller decrease in the tumor load would indicate a relatively less efficacious cancer therapy.

If the tumor load at the first time point is lower than the tumor load at the second time point, then the cancer therapy is not effective in treating cancer in the subject. Also, the magnitude of increase would indicate how ineffective is the cancer therapy. A bigger increase in the tumor load would indicate an ineffective cancer therapy, whereas a relatively smaller increase in the tumor load would indicate a mildly effective cancer therapy.

The single molecule sequencing of cfDNA can be optimized according to the methods disclosed herein. Particularly, in some cases, sequencing the sample of cfDNA comprises producing a cfDNA sequencing library, comprising:
producing an A-tailed cfDNA by incubating the cfDNA with an end-repair and A-tailing enzyme mix comprising of a DNA kinase, a blunting enzyme, and DNA polymerase for at least 30 minutes, and
ligating a sequencing adapter to the A-tailed cfDNA by incubating the A-tailed cfDNA with the sequencing adapter in the presence of a DNA ligase for at least 4 hours at about 20° C. thereby producing the DNA sequencing library.

Higher yield for single molecule sequencing is achieved using the methods disclosed herein. The end-repair and A-tailing steps are performed in the conventional protocol for a shorter period of time, e.g., about 10 minutes. Instead, in the methods disclosed herein, producing end-repaired and A-tailed cfDNA comprises incubating the cfDNA with an end-repair and A-tailing enzyme mix for at least 30 minutes.

Also, compared to conventional protocol, ligation steps are performed for a significantly longer period of time in the methods disclosed herein. Particularly, in the conventional protocol, ligation is performed for about 10 minutes, whereas in the methods disclosed herein ligating a sequencing adapter to the A-tailed cfDNA comprises incubating the A-tailed cfDNA with the sequencing adapter in the presence of a DNA ligase for at least 4 hours. The temperature of incubation can be between 15° C. to 25° C., particularly, at about 20° C.

Longer durations of A-tailing and ligation steps in the methods disclosed herein results in an increase in the aligned reads of at least about 2 fold, particularly, about 2 fold to 10 fold.

In some cases, multiple samples are pooled in the sequencing step by multiplexing the cfDNA. For example, barcoded adapters can be ligated to cfDNA sequencing library to produce a multiplexed cfDNA sequencing library. Thus, in some cases, the method of producing a cfDNA sequencing library further comprises producing a multiplexed cfDNA sequencing library, the method comprising:
producing an A-tailed cfDNA sequencing library by incubating individual cfDNA samples with an end-repair and A-tailing enzyme mix comprising of a DNA kinase, a blunting enzyme, and DNA polymerase, and
ligating a barcoded multiplexing adapter to the A-tailed cfDNA sequencing library thereby producing the multiplexed cfDNA sequencing library,
pooling the individual barcoded cfDNA libraries together,
producing a pooled A-tailed cfDNA sequencing library by incubating the pooled library with a second end-repair and A-tailing enzyme mix comprising of a DNA kinase, a blunting enzyme, and
ligating a a sequencing adapter to the A-tailed pooled cfDNA library by incubating the A-tailed cfDNA with the sequencing adapter in the presence of a DNA ligase for at least 1 hour at about 20° C. thereby producing the DNA sequencing library.

Any suitable DNA polymerase can be used in the A-tailing steps. Certain non-limiting DNA polymerases include Taq DNA polymerase or Klenow fragment.

Similarly, any suitable DNA ligase can be used in the ligation steps. A non-limiting DNA ligase includes T4 DNA ligase.

The optimized library preparation disclosed herein allows using lower amounts of initial cfDNA used to prepare the cfDNA library. Particularly, the amount of cfDNA used in producing the A-tailed cfDNA is between 100 μg and 5 ng, between 800 ng and 1.5 ng, or about 1 ng.

### Utility

The methods described in this disclosure find use in a variety of applications. Applications of interest include, but are not limited to: research applications and therapeutic applications. Methods of the disclosure find use in a variety of different applications including any convenient application where identifying methylation profiles of cfDNA is desired.

For example, the method finds particular use in detecting the presence of tDNA in cfDNA samples obtained from a subject.

Tumor load calculated according to the methods disclosed herein can be used to monitor the progression of a cancer in a subject. For example, increasing tumor load can indicate advancing disease, whereas decreasing tumor load can indicate cancer remission.

Tumor load can also be used to monitor efficacy of a cancer therapy administered to a subject. For example, increasing tumor load can indicate that a cancer therapy is not effective, whereas decreasing tumor load can indicate that a cancer therapy is effective.

The methods disclosed herein are exemplified based on analysis of methylation status of CpG sites in the genome; however, additional epigenetic modifications are known in the art to be associated with disease development and progression. Therefore, the methods disclosed herein can also be applied to analyzing such additional epigenetic modifications to diagnose and monitor cancer as well as other diseases.

Moreover, the methods disclosed herein are exemplified for use in cancer diagnosis, cancer progression monitoring, or cancer therapy monitoring. However, these methods can be used for diagnosing or monitoring any disease where epigenetic modification at differentially modified sites can be used to identify molecules of cfDNA that originate from disease causing cells versus normal cells. Similarly, these methods can be used for identifying molecules of cfDNA in a pregnant mother that originate from the mother's cells versus the cells of the fetus.

In some cases, the methods disclosed herein can also be applied in diagnosis and monitoring of diseases where the methylation status of a target locus is associated with a disease. Such diseases include liver diseases such as chronic hepatitis or cirrhosis, neuropsychiatric disorders caused by epigenetic factors, Crohn's disease, autoimmune disorders, such as systemic lupus erythematosus (SLE), rheumatoid arthritis (RA), systemic sclerosis (SSc), Sjogren's syndrome (SS), autoimmune thyroid diseases (AITD), and type 1 diabetes (T1D). Additional such diseases are well known in the art and diagnosis and monitoring of such diseases is within the purview of the disclosure.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Materials and Methods

### Clinical Samples

Informed consent was obtained based on a protocol that was approved by Stanford University's Institutional Review Board. Blood and tissue samples came from the Stanford Tissue Bank and the Stanford Blood Center. Whole blood from the Stanford Cancer Center was obtained from patients in Streck or EDTA tubes before receiving as plasma in cryovials. Colorectal tumor tissue was archived by flash freezing in liquid nitrogen and stored at −80° C. Plasma from the Stanford Tissue Bank was obtained as single aliquots in 1 ml cryovials. Where noted, frozen tumor samples and matched plasma were also obtained. Whole blood was obtained from anonymous donors from the Stanford Blood Center for healthy controls, which was then centrifuged into plasma and buffy coat fractions. Tissue and plasma were stored at −80° C. before processing.

### DNA Extraction

Extracted DNA was obtained from tissue biopsies using the Maxwell 16 DNA extraction kit (Promega). Briefly, a small fragment of the tissue was excised from the tissue sample with a scalpel and deposited into the input well of the DNA purification cartridge. The cartridge was placed into the Maxwell 16 instrument (Promega), and the associated protocol was run. For extracting cell-free DNA, plasma was separated from whole blood by centrifugation. The plasma fraction was pipetted into a Maxwell 16 ccfDNA Plasma kit cartridge (Promega) using the standard instrument protocol. The cellular blood portion was extracted using a Maxwell 16 LEV Blood DNA Kit. Yields were measured by Qubit (Thermo Fisher Scientific). Cell-free DNA was quantified using the AccuBlue NextGen DNA Quantification Kit (Biotium).

### Nucleosome DNA Controls

To generate DNA fragments modeling the qualities of cell-free DNA, the EZ Nucleosomal DNA Prep Kit (Zymo Research) was used. This method uses DNAse to digest open chromatin positions and yields a fragment pattern characteristic of cell-free DNA instead of random fragmentation. Briefly, nuclei were processed from whole cells by the addition of a nuclei prep buffer that lyses the cell membrane but leaves the nuclei membrane intact. Enzymatic DNAse digestion then fragments DNA at unprotected locations, after which DNA is purified with the kit's included components. For nucleosomes from cancer cell lines, adherent cells treated with trypsin were used.

Peripheral blood mononuclear cells (PBMCs) were used for nucleosomes representing healthy controls from healthy donors obtained from the Stanford Blood Center. Whole blood was diluted with an equal volume of PBS and added to a SepMate PBMC isolation tube (STEMCELL Technologies) containing Ficoll. The tube was spun at 1200 g for 10 minutes before decanting into a new tube. Cells spun again at 400 g for 5 minutes and washed with PBS before resuspending in freezing medium (90% FBS/10% DMSO). Isolated PBMCs were then used as input for the nucleosome preparation kit. Admixtures were generated by diluting PBMC and cancer cell line nucleosomes to a target concentration (e.g. 1 ng/μl) and then mixing to known ratios. Serial dilutions of this mixture are then performed to simulate lower input amounts.

### Sequencing Library Preparation

An optimized protocol was developed for generating sequencing libraries that accommodate the low input amounts of cfDNA and maximize sample barcode adapters' incorporation rate. Briefly, 25 μl of extracted cfDNA (out of a typical 50 pl extracted volume) was diluted with 25 μl of water. The sample DNA underwent End-Repair and A-tailing with conditions of 20° C. for 30 minutes and 65° C. for 30 minutes (Roche KAPA HyperPrep kit). Native barcodes were ligated using 5 μl of each barcoded adapter (EXP-NBD196, Oxford Nanopore Technologies) following the standard reaction volumes in the KAPA HyperPrep workflow. A thermocycler was used to ligate for 4.5 hours at 20° C. before holding at 4° C. overnight to increase the ligation yield. These steps provided a higher ligation rate of cell-free DNA molecules to a native barcode adapter than the standard protocol's shorter End-Repair/A-tailing and ligation time (10 minutes for each step).

After the ligation step, 88 μl of Mag-Bind Total NGS beads (Omega Bio-Tek; an alternative to Ampure XP beads) were added and mixed to each reaction. After incubation for 5 minutes, the mixtures were pooled together into a 50 μl centrifuge tube. The beads were magnetized and washed with 80% ethanol using a DynaMag separation rack (Thermo Fisher Scientific) before eluting in 600 μl of 10 mM Tris-HCl pH 8.0 buffer. A second bead cleanup step was performed with 900 μl Mag-Bind Total NGS beads (1.5× ratio) and the same magnetic rack procedure. The elution solution was 50 pl 10 mM Tris-HCl pH 8.0 buffer.

Commercially supported multiplexing on the Oxford Nanopore platform is restricted to the AMII adapter, which has the same motor protein family as the LSK109 chemistry. The disadvantage is the active burning of on-chip “fuel” when molecules are not sequencing leading to rapid flow cell exhaustion. To address this, the library preparation process was modified to use the updated “fuel-fix” adapter (LSK110 kit, Oxford Nanopore Technologies) is not out-of-the-box compatible with any native barcoding kit. Some new steps were developed to facilitate this multiplexing. A second End-Repair and A-tailing reaction was performed using the Kapa HyperPrep library preparation kit to remove the sticky end from barcode multiplexing adapter. For the ligation step, an increased amount (10 μl) of the AMX-F adapter (LSK110, Oxford Nanopore Technologies) was used to maximize the amount of sequenced ligated fragments. This second ligation reaction occurred for 1.5 hours. Then 88 μl of Mag-Bind was mixed with Total NGS beads and incubated for 5 minutes. As in the standard protocol, the beads were washed with 200 pl SFB buffer (Oxford Nanopore Technologies) with gentle flicking of the tube to resuspend the beads during the wash steps. The beads were resuspended in EB buffer (Oxford Nanopore Technologies). 1 μl was used for quantification with Qubit (Oxford Nanopore Technologies) and 1 μl was used for determining the DNA size with an E-gel EX cartridge (Thermo Fisher Scientific).

For tissue samples, 1-2 ug of extracted DNA or the maximum amount of extracted material where possible was used. The standard Kapa HyperPrep library preparation kit protocol was followed using 5 μl of AMX-F adapter (LSK110) without barcoding. Each sample was loaded into its own PromethION flow cell for sequencing.

For comparison with the standard library preparation protocol, the standard protocol was followed for Native Barcoding (EXP-NBD196) coupled with the SQK-LSK109 library preparation kit using the AMII adapter.

### Example 1—Single cfDNA Molecule Sequencing and Single Read Classification

cfDNA was sequenced from 20 patients with colorectal cancer. The sequence data yield ranged from one million to 72 million reads per sample. A fluorometric assay was used to orthogonally quantify each cfDNA sample; the fluorometric measurements highly correlated with the sequencing yield (FIG. 1C). Alongside the cancer patient-derived cfDNA, cfDNA derived from several healthy blood donors was also sequenced—this dataset provided a background control determining changes in methylation and size distribution (FIG. 1D). Overall, several distinct differences were observed between the patient-derived and healthy cfDNA. First, the average global methylation differed by as much as 5% in some patient-derived cfDNA, indicative of global epigenetic reprogramming (FIG. 1E). In one patient's cfDNA, the average genome-wide methylation dropped from an average of about 65% in the healthy controls to less than 50% indicative of massive genome-wide hypomethylation. Using a cutoff of 250 bp between mono- and di-nucleosome fragment sizes, patient-derived cfDNA was observed to be highly enriched in mononucleosomes by approximately a factor of two (FIG. 1F). Finally, multiple testing-corrected significance testing of gene-level methylation averages yielded hundreds of features that differ between healthy and patient-derived cfDNA (FIG. 1G). Consistent with increasing amounts of cfDNA shed from epithelial tumors, an increase was observed in the methylation of immunologic genes such as CD79A, and a decrease was observed in methylation of a tumorigenic modulation gene DICER1. Gene list enrichment analysis yielded significant hits in the Myc pathway (FIG. 5), suggesting that the observed changes in gene-level methylation are cancer-specific.

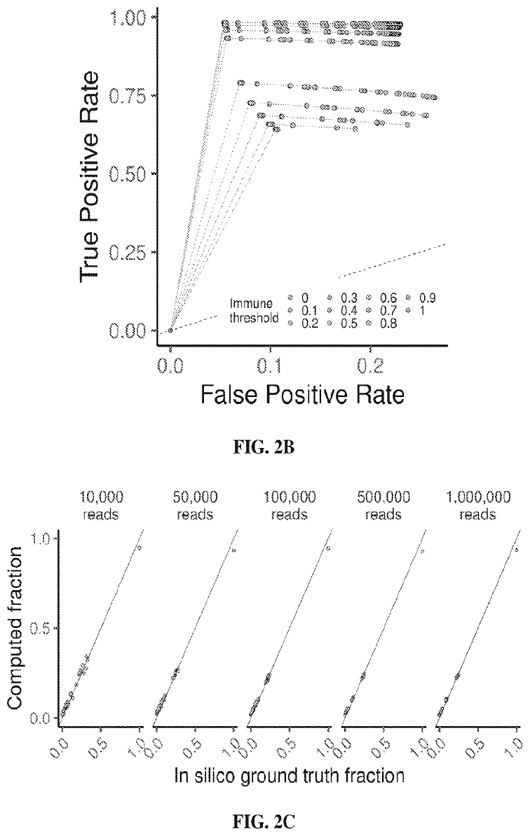

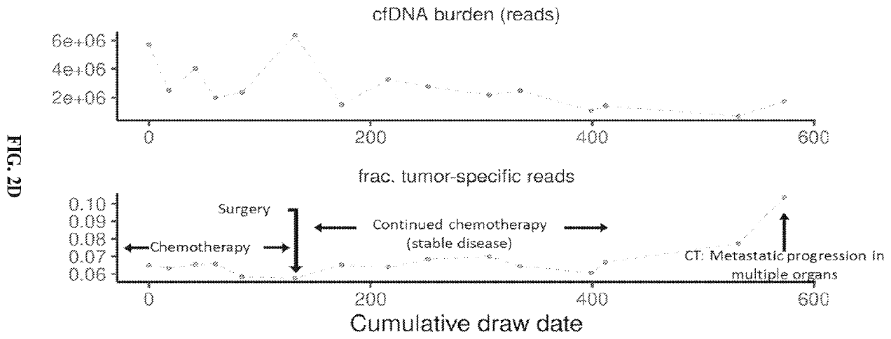

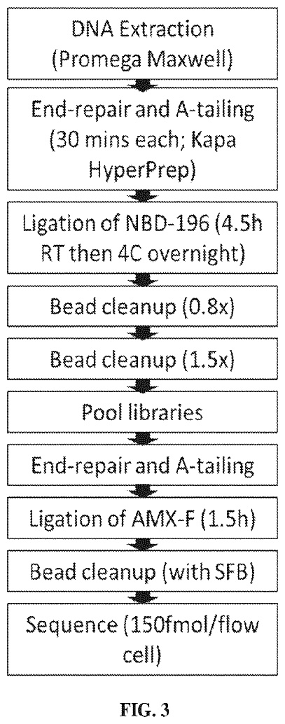

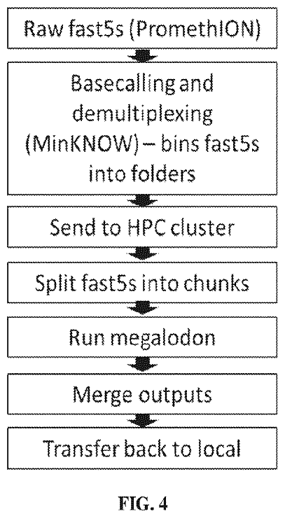

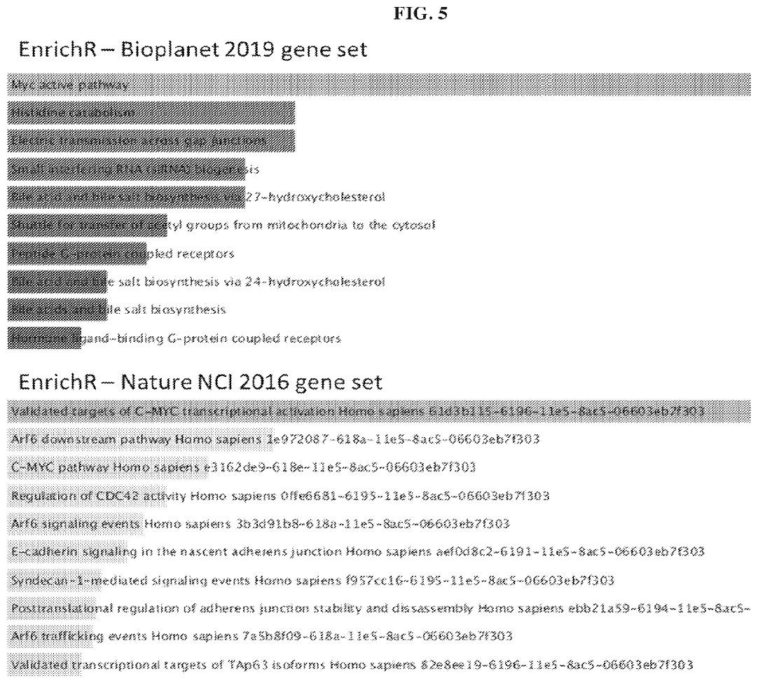

Next, it was determined whether single cfDNA molecules can be classified on a read-by-read basis. Rather than using aggregate methylation profiles, single-molecule determination of nucleotide modification coupled with a highly matched dataset of known tumor-specific methylation sites (such as in a primary tumor) could be used for determining disease progression and residual disease burden (FIG. 2A). Briefly, methylation profiles from individual single molecule reads can be scored by counting the proportion of matching methylation sites against a reference profile for every read. A dual-threshold score was used to stringently classify reads as being immune- or cancer-derived (FIG. 6), with reads with scores in between as not having a confident classification and thus discarded. To validate this approach, in silico admixtures were generated between donor PBMC nucleosomes from a healthy donor and the GP2D cancer cell line and classification accuracy was measured against the ground truth (FIG. 2B). The overall accuracy was determined to be over 90% when using stringent thresholds for classification of immune-derived versus cancer-derived cfDNA, with a maximum AUC of 0.969 with an appropriate choice of thresholds (FIG. 7). As a trade-off, using the most stringent cutoff criteria, the proportion of reads that can be confidently classified can be as low as 25% of reads intersecting with the reference profiles of interest (FIG. 6).

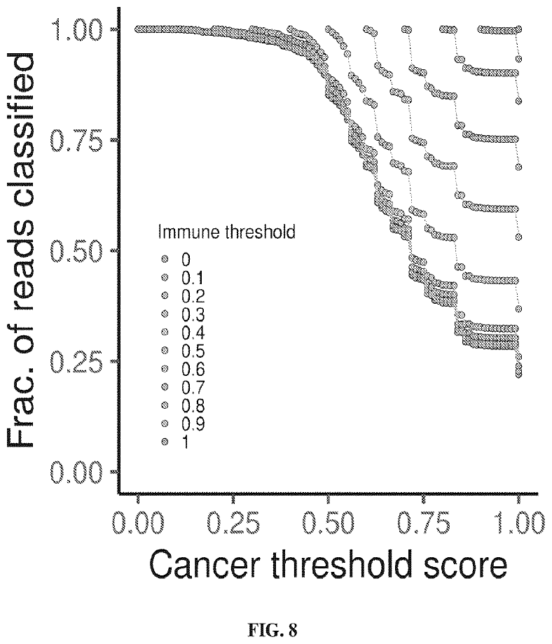

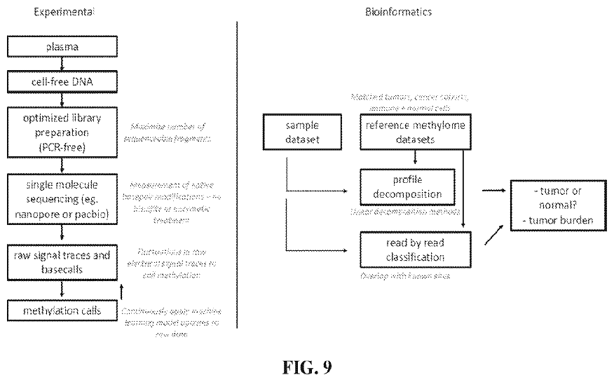

As further validation, a set of experimental admixtures was developed where GP2D nucleosome DNA was added to donor nucleosome DNA, whilst also varying the total quantity of DNA in the reaction. Overall, a corresponding increase was observed in cancer-derived reads at higher GP2D admixing fractions (FIG. 8); the limit of detection sensitivity was largely limited to the input amount, as a single nanogram to sub-picogram amounts of starting material would yield less than 300 genome equivalents, assuming perfect reaction efficiency.

Longitudinal cfDNA samples as well as their associated primary tumors and peripheral blood from cancer patients with various gastrointestinal cancers were also sequenced. The reference profiles, intersected together, yielded over one million CpG sites per patient. The longitudinal dynamics were determined of tumor burden through single-molecule cfDNA classification (FIG. 2A). Changes were further annotated in tumor-derived DNA content over the course of treatment. Importantly, increases observed in tumor burden correlated with clinically notable events. For example, the fraction of tumor-specific reads and overall cfDNA burden was tracked over the course of treatment for a metastatic colorectal patient for almost 600 days (FIG. 2D). The fraction of cfDNA changed over the course of treatment, and increased over time coinciding with substantial multi-organ metastatic progression.

### Example 2—Single Molecule Sequencing and Data Processing

Sequencing was performed on the Oxford Nanopore Technologies' PromethION 24 instrument. The entire library volume was used for a given sequencing run for cell-free DNA pools. Approximately 150 fmol of the library was loaded per flow cell. For tissue samples, one entire flow cell per sample was used. Sequencing runs had a duration of 72 hours. Barcode demultiplexing was performed on the sequencer using onboard base-calling in MinKNOW with the “high accuracy” model and then transferred to a separate storage device. Raw fast5 sequencing data were processed using Megalodon v2.4.0 (Oxford Nanopore Technologies) and Guppy (v5.0.16) with the “dna_r9.4.1_450bps_modbases_5mc_hac_prom.cfg” model for each demultiplexed barcode folder with standard settings. The GRCh38 reference was used for alignment. The output consists of a file in BedMethyl format for each sample. The files included modified base calls, a sequencing alignment bam file with modified base calls for each read, and a per-read text file containing modified base call probabilities. The BedMethyl and sequence-alignment bam files were sorted and indexed with samtools before further processing. In cases of large quantities of samples (e.g. from multiple flow cells and many barcodes), data was transferred to the Sherlock High-Performance Computing cluster at Stanford University for massively parallel data processing.

The overall methylation of sequenced cfDNA was determined by taking the average of all methylation values across all sequenced sites (coverage >0). For determination of nucleosome enrichment, the estimated fragment size was tabulated as inferred by the alignment length, and set a cutoff of 250.5 base pairs separating mononucleosomal and dinucleosomal states. This was then compiled for all reads and all samples sequenced.

To determine gene-level methylation for all sequenced cfDNA samples, average methylation profiles were determined for each “gene”-level annotation in GENCODE v38. These were then filtered to exclude annotations that were pseudogenes, unprocessed, “to be experimentally conformed,” lncRNAs, and miRNAs. To determine statistically significant differences in gene-level methylation, a t-test was used to compare methylation between the healthy donor-derived cfDNA and cancer patient-derived cfDNA, with fdr-based multiple testing correction. A cutoff of q<0.01 was used.

### Example 3—In Silico Mixture Experiments

To simulate ctDNA data of varying fractions, in silico admixtures were generated of sequence data from the GP2D cancer cell line-derived and PBMC-derived nucleosomes. Using a Python script, two sequence-aligned bam files were mixed using a known random seed to ensure reproducibility. The number of reads was controlled to simulate read depth. Methylation profiles were compiled from the Mm and Ml tags using the modbampy library as part of the modbam2bed package (https://github.com/epi2me-labs/modbam2bed). Only the methylated reads were used that mapped to the reference and the subsequent bam file was used for downstream analysis. The remainders of the reads were not used including unmapped reads and those with secondary or supplementary alignments. As another output, the metadata about the sample origins was included; namely whether it originated from PBMC-derived nucleosomes or from a cancer cell line.

### Example 4—Methylation Read Classification

A Python-based computational workflow was built to classify whether an individual read is associated with an associated reference methylation profile. This process starts with the sequence-aligned bam file containing read modifications (from megalodon). First, each individual read was classified alongside a reference methylome containing informative methylation sites. This process generated a value fᵢ^{tissue} where i is the read number from 1 to the total number of aligned reads.

$$Specifically , {f}_{i}^{tissue} = {\sum }_{i} p ( {m}_{i} = {m}_{i}^{′} ) / {N}_{sites} = \{ \begin{matrix}{m}_{i}^{′} & if {m}_{i} = 1 \\ 100 - {m}_{i}^{′} & if {m}_{i} = 0\end{matrix} .$$

To implement this scheme, the methylation status (mᵢ . . . mₙ) of each CpG site for each read from a given sample, as well as its reference coordinate were obtained. Then these coordinates of the CpG sites were intersected to the corresponding locations of a candidate tissue reference methylation profile (e.g. from immune cells or matched primary tumor, with methylation profile mᵢ′ . . . mₙ′). Subsequently, a matching score was calculated, where each site is scored mᵢ′ if the mᵢ is methylated; otherwise, it is scored 100−mᵢ′. In other words, the score is the probability that the methylation site and value mᵢ is the same as the reference profile site mᵢ′, which is equivalent to the reference profile's methylation level at that site. It is then divided by the total number of candidate CpG sites on the read to derive fᵢ^{tissue}. Reads with no candidate CpG sites or matching locations in a reference methylome are not considered. A per-read tumor score is then assigned by the ratio of scores pᵢ=fᵢ^{tumor}/(fᵢ^{tumor}+fᵢ^{immune}), with scores close to zero indicating likely matches to immune cells, and scores close to one indicating likely matches to tumor tissue. A final classification is determined by setting thresholds for matching to immune and cancer methylation profiles. By using a dual threshold system, a subset of reads in between the two thresholds cannot be definitively classified and are thus not called to be of either type. These reads are excluded from the final analysis. The two thresholds were used to determine ROC curves and AUC performance metrics.

### Example 5—Methylation Profile Processing of Primary Tumors

For a subset of patient samples, the primary tumor and matched normal tissue underwent single molecule sequencing; methylation calls were also performed with megalodon. R script was used to read both the tumor and immune methylation profiles, while intersecting only on sites with coverage greater than four in both samples. For the immune profile, the methylation profile of a healthy donor from the Stanford Blood Center was used. A site was considered to be methylated if the percentage methylation per a given genomic segment was greater than zero. The resultant table was used for read-level classification by using the methylation profile matching scheme shown above. Clinical events were recorded alongside each time point.

Overall, single molecule sequencing of cell-free DNA analytes for identifying methylation was demonstrated. Despite the overall sequencing yield being orders of magnitude below what is achievable with Illumina sequencing, single molecule sequencing offered significant advantages compared to short-read approaches. Measuring DNA methylation with Illumina sequencing requires extensive sample manipulation, amplification, and bioinformatic processing. The methods disclosed herein demonstrated that streamlined methylation analysis of cell-free DNA is feasible with significantly fewer experimental procedures and bottlenecks. As single molecule, cell-free DNA methylation determination is only dependent on machine learning models rather than on experimental manipulation of unmethylated residues, newer models can be applied to archived raw data to incorporate the detection of other modified bases. As methylation is generally anti-correlated with gene expression, this type of methylation profile can help determine tumor origins and subtypes. In summary, using this sequencing method significantly expands epigenomic analysis of cell-free DNA, which can have a significant impact on liquid biopsy diagnostics for cancer detection.

1. A method for detecting a molecule of tumor DNA (tDNA) in a sample of cell-free DNA (cfDNA), the method comprising:
sequencing the sample of cfDNA using a single molecule sequencing to obtain sequence reads;
analyzing a sequence read by:
(a) identifying in the sequence read differentially methylated CpG sites, the differentially methylated CpG sites having different methylation status in a cancer cell versus a non-cancer cell,
(b) determining which differentially methylated CpG sites in the sequence read are methylated and which differentially methylated CpG sites are unmethylated to obtain a methylation profile for the sequence read;
(c) calculating a first methylation score based on: i) the number of differentially methylated CpG sites in the sequence read that matches the methylation status of the differentially methylated CpG sites in the cancer cell and ii) the total number of differentially methylated CpG sites in the sequence read,
(d) calculating a second methylation score based on: i) the number of differentially methylated CpG sites in the sequence read that matches the methylation status of the differentially methylated CpG sites in a non-cancer cell and ii) the total number of differentially methylated CpG sites in the sequence read,
(e) identifying the sequence read as being from a molecule of tDNA based the scores calculated in steps (c) and (d).

2. The method of claim 1, further comprising:
(f) identifying the fragment size of a single molecule sequence read as being from a mono-nucleosome or di-nucleosome or higher size range,
(g) aggregating the fragment sizes and nucleosome classification across all cfDNA reads in a sample,
(h) comparing the ratio of mono-nucleosomes to di-nucleosome sequence counts in a sample that of a reference cohort; and
(i) determining whether the cfDNA sample as being similar to cancer or healthy cfDNA.

3. The method of claim 1, wherein the single molecule sequencing is performed by nanopore sequencing.

4. The method of claim 1, wherein the single molecule sequencing is performed by single molecule real-time (SMRT) sequencing.

5. The method of claim 1, wherein sequencing the sample of cfDNA comprises producing a cfDNA sequencing library, comprising:
producing an A-tailed cfDNA by incubating the cfDNA with an end-repair and A-tailing enzyme mix comprising of a DNA kinase, a blunting enzyme, and DNA polymerase for at least 30 minutes, and
ligating a sequencing adapter to the A-tailed cfDNA by incubating the A-tailed cfDNA with the sequencing adapter in the presence of a DNA ligase for at least 4 hours at about 20° C. thereby producing the cfDNA sequencing library.

6. The method of claim 5, wherein said producing the cfDNA sequencing library further comprises producing a multiplexed cfDNA sequencing library, the method comprising:
producing an A-tailed cfDNA sequencing library by incubating the cfDNA sequencing library with an end-repair and A-tailing enzyme mix comprising of a DNA kinase, a blunting enzyme, and second DNA polymerase,
ligating a barcoded multiplexing adapter to the A-tailed cfDNA sequencing library,
pooling multiple barcoded cfDNA libraries together,
producing a pooled A-tailed cfDNA sequencing library by incubating the pooled cfDNA sequencing library with a second end-repair and A-tailing enzyme mix comprising of a DNA kinase, a blunting enzyme, and
ligating a sequencing adapter to the pooled A-tailed cfDNA sequencing library, thereby producing the multiplexed cfDNA sequencing library.

7. The method of claim 5, wherein the first and/or the second DNA polymerase is Taq DNA polymerase or Klenow fragment.

8. The method of claim 5, wherein the DNA ligase is T4 DNA ligase.

9. The method of claim 5, wherein the amount of cfDNA used in producing the A-tailed cfDNA is between 400 μg and 2 ng.

10. The method of claim 4, further comprising sequencing the cfDNA sequencing library by nanopore sequencing.

11. The method of claim 5, further comprising sequencing the cfDNA sequencing library by SMRT sequencing.

12. The method of claim 1, further comprising estimating the number of molecules of tDNA in the sample of cfDNA.

13. The method of claim 12, further comprising estimating as a tumor load of the cfDNA the proportion of the number of molecules of tDNA in the cfDNA sample.

14. A method of monitoring a cancer progression in a subject, the method comprising:
estimating according to claim 13 the tumor load of cfDNA in the subject at a first time point and a later second time point.

15. A method of determining efficacy of a cancer therapy administered to a subject, the method comprising:
estimating according to claim 13 the tumor load of cfDNA in the subject at a first time point and a later second time point.

16. The method of claim 15, wherein the cancer therapy is administered before the first time point.

17. The method of claim 15, wherein the cancer therapy is administered after the first time point and before the second time point.
